(19) United States
(12) Patent Application Publication
(10) Pub. No.: US 2026/0188516 A1
(43) Pub. Date: Jul. 2, 2026

(54) APPARATUS AND METHOD FOR PROVIDING GUIDE INFORMATION FOR REHABILITATION EXERCISE BASED ON ARTIFICIAL INTELLIGENCE

(71) Applicant: ORTHOCARE CO., LTD., Seoul (KR)

(72) Inventors: Jong Ho KIM, Seoul (KR); Yang Soo KIM, Seoul (KR); Youn Kyu LEE, Seoul (KR); Dong Hwan LEE, Seoul (KR); Se Won LEE, Seoul (KR); Ji Hyun RYU, Seoul (KR); Jin Hyung IM, Seoul (KR)

(73) Assignee: ORTHOCARE CO., LTD., Seoul (KR)

(21) Appl. No.: 19/539,969

(22) Filed: Feb. 13, 2026

Related U.S. Application Data
(63) Continuation of application No. PCT/KR2024/007663, filed on Jun. 4, 2024.

(30) Foreign Application Priority Data
Aug. 17, 2023 (KR) 10-2023-0107745

(51) Int. Cl. G16H 50/30; G06V 10/82; G06V 40/10; G06V 40/20; G16H 20/30; G16H 30/40; G16H 50/20; G16H 50/70

(52) U.S. Cl. CPC G16H 50/30; G06V 10/82; G06V 40/10; G06V 40/23; G16H 20/30; G16H 30/40; G16H 50/20; G16H 50/70

(57) ABSTRACT

The present disclosure provides an apparatus and method for providing guide information for surgical planning and rehabilitation exercise based on artificial intelligence, capable of predicting a postoperative complication at a surgical site of a subject patient based on artificial intelligence and providing prediction information to medical staff or a medical institution so that, when performing surgery on the subject patient or when the subject patient performs postoperative rehabilitation exercise, surgery and postoperative rehabilitation are performed in consideration of a likelihood of postoperative complication, thereby lowering a probability of occurrence of a complication during a postoperative rehabilitation period.

### CROSS-REFERENCE TO RELATED APPLICATIONS

The present application is a continuation of International Patent Application No. PCT/KR2024/007663, filed on Jun. 4, 2024, which is based upon and claims the benefit of priority to Korean Patent Application No. 10-2023-0107745 filed on Aug. 17, 2023. The disclosures of the above-listed applications are hereby incorporated by reference herein in their entirety.

### BACKGROUND

### 1. Technical Field

The present disclosure relates to an apparatus and method for providing guide information, and more particularly, to an apparatus and method for providing guide information for rehabilitation exercise based on artificial intelligence.

### 2. Description of Related Art

It is known that tens of thousands to hundreds of thousands of surgical treatments are performed nationwide each year for each orthopedic disease, and the frequency is increasing exponentially with population aging. In orthopedic surgery, it is necessary to protect a joint for a predetermined period using a cast, a splint, an orthosis, or the like to protect a surgical site, and in such cases, a problem of joint stiffness occurs. Even in surgery that does not involve joint protection, joint stiffness easily occurs after surgery because inflammatory reactions occur rapidly in joints of the human body.

However, treatment for joint stiffness inevitably accompanying orthopedic surgery is entirely the patient's responsibility, and in reality, the patient learns an exercise method by themselves through a video or a pamphlet and then proceeds with self-rehabilitation exercise in an improvised manner.

In addition, when rehabilitation is not properly performed because persistent pain continues or because the patient is not confident that their exercise progress is proceeding correctly, a vicious cycle may be repeated in which the patient visits a hospital at a late stage to receive rehabilitation therapy or manual therapy.

Accordingly, there is a need to develop a technology that enables a patient to perform rehabilitation exercise more efficiently and systematically after orthopedic surgery.

### SUMMARY

The present disclosure provides an apparatus and method for providing guide information for surgical planning and rehabilitation exercise based on artificial intelligence, capable of predicting a postoperative complication at a surgical site of a subject patient based on artificial intelligence and providing prediction information to medical staff or a medical institution so that, when performing surgery on the subject patient or when the subject patient performs postoperative rehabilitation exercise, surgery and postoperative rehabilitation are performed in consideration of a likelihood of postoperative complication, thereby lowering a probability of occurrence of a complication during a postoperative rehabilitation period.

Meanwhile, the present disclosure provides an apparatus and method for providing guide information for rehabilitation exercise based on artificial intelligence, capable of enabling a patient to check (self-check) an individual condition based on artificial intelligence and providing customized guide information corresponding to the condition so that the patient can perform rehabilitation exercise more efficiently and systematically.

The technical problems to be solved by the present disclosure are not limited to the above-mentioned technical problems, and other technical problems not mentioned herein will be clearly understood by those skilled in the art from the following description.

An apparatus for providing guide information for rehabilitation exercise based on artificial intelligence according to an aspect of the present disclosure may include: a communication module configured to perform communication with an external device; a storage module configured to store at least one process for providing guide information for rehabilitation exercise based on artificial intelligence; and a control module configured to perform an operation for providing guide information for rehabilitation exercise based on artificial intelligence based on the at least one process, and the control module may be configured to: collect, based on the storage module and a separate database, medical data of a subject patient and medical images captured before surgery; perform preprocessing on the medical images, and input the preprocessed medical images to a first artificial-intelligence-based pre-trained model to predict a probability of occurrence of a postoperative complication; and generate prediction information based on the predicted result and provide the prediction information to a medical staff terminal.

Meanwhile, a method for providing guide information for rehabilitation exercise based on artificial intelligence according to an aspect of the present disclosure may include: collecting, based on a storage module and a separate database, medical data of a subject patient and medical images captured before surgery; performing preprocessing on the medical images; and inputting the preprocessed medical images to a first artificial-intelligence-based pre-trained model to predict a probability of occurrence of a postoperative complication, generating prediction information based on the predicted result, and providing the prediction information to a medical staff terminal.

In addition, a computer program stored in a computer-readable recording medium for executing a method for implementing the present disclosure may be further provided.

In addition, a computer-readable recording medium storing a computer program for executing a method for implementing the present disclosure may be further provided.

### BRIEF DESCRIPTION OF THE FIGURES

FIG. 1 is a diagram illustrating a system for providing guide information for rehabilitation exercise based on artificial intelligence according to an embodiment of the present disclosure.

FIG. 2 is a diagram illustrating a configuration of an apparatus for providing guide information for rehabilitation exercise based on artificial intelligence according to an embodiment of the present disclosure.

FIG. 3 is a diagram illustrating a method for providing guide information for rehabilitation exercise based on artificial intelligence according to an embodiment of the present disclosure.

FIG. 4 is a diagram illustrating a method of performing training for a first artificial-intelligence-based pre-trained model according to an embodiment of the present disclosure.

FIG. 5 is a diagram illustrating a series of operations for preprocessing at least one medical image for each of other patients collected according to an embodiment of the present disclosure.

FIG. 6 is a diagram illustrating a method of analyzing a motion of a subject patient through a second artificial-intelligence-based pre-trained model according to an embodiment of the present disclosure.

FIG. 7 is a diagram illustrating a method of performing training for a second artificial-intelligence-based pre-trained model according to an embodiment of the present disclosure.

FIG. 8 is a diagram illustrating a series of operations for preprocessing at least one rehabilitation exercise video for each of other patients collected according to an embodiment of the present disclosure.

### DETAILED DESCRIPTION

Advantages and features of the present disclosure, and methods for achieving them, will become clear with reference to the embodiments described in detail below in conjunction with the accompanying drawings. However, the present disclosure is not limited to the embodiments disclosed below but may be implemented in various different forms; rather, these embodiments are provided so that this disclosure will be thorough and complete and will fully convey the scope of the disclosure to those skilled in the art, and the present disclosure is only defined by the scope of the claims.

The terminology used herein is for the purpose of describing embodiments and is not intended to be limiting of the present disclosure. In the present specification, the singular forms include the plural forms as well unless the context clearly indicates otherwise. The terms “comprises” and/or “comprising” used in the specification do not exclude the presence or addition of one or more other components in addition to the mentioned components. Like reference numerals refer to like components throughout the specification, and “and/or” includes each and every combination of one or more of the mentioned components. Although “first”, “second”, etc. are used to describe various components, these components are, of course, not limited by these terms. These terms are only used to distinguish one component from another component. Therefore, it goes without saying that a first component mentioned below may be a second component within the technical spirit of the present disclosure.

Unless otherwise defined, all terms (including technical and scientific terms) used herein may be used with meanings commonly understood by those skilled in the art to which the present disclosure belongs. In addition, terms defined in commonly used dictionaries are not interpreted ideally or excessively unless explicitly and specially defined.

Throughout the present disclosure, the same reference numerals refer to the same components. The present disclosure does not describe all elements of the embodiments, and common content in the technical field to which the present disclosure belongs or redundant content between embodiments will be omitted. The term “unit” or “module” used in the specification refers to a hardware component such as software, FPGA, or ASIC, and “unit” or “module” performs certain roles. However, “unit” or “module” is not limited to software or hardware. “Unit” or “module” may be configured to reside in an addressable storage medium or may be configured to reproduce one or more processors. Thus, as an example, “unit” or “module” includes components such as software components, object-oriented software components, class components, and task components, processes, functions, attributes, procedures, subroutines, segments of program code, drivers, firmware, microcode, circuits, data, databases, data structures, tables, arrays, and variables. Functions provided within components and “units” or “modules” may be combined into a smaller number of components and “units” or “modules” or may be further separated into additional components and “units” or “modules”.

Throughout the specification, when a part is said to be “connected” to another part, this includes not only a case where it is directly connected but also a case where it is indirectly connected, and indirect connection includes connection through a wireless communication network.

In addition, when a part is said to “include” a component, this means that other components are not excluded but may be further included unless specifically stated otherwise.

Throughout the specification, when a member is said to be disposed “on” another member, this includes not only a case where the member is in contact with the other member but also a case where another member exists between the two members.

The terms first, second, etc. are used to distinguish one component from another component, and the components are not limited by the aforementioned terms.

Singular expressions include plural expressions unless the context clearly indicates otherwise.

In each step, identification codes are used for convenience of explanation, and the identification codes do not describe the order of each step, and each step may be performed differently from the specified order unless a specific order is clearly described in context.

Terms used in the following description are defined as follows.

In the present specification, a first artificial-intelligence-based pre-trained model and a second artificial-intelligence-based pre-trained model are deep-learning-based prediction models, and through each of them, a re-rupture rate for a surgical site of a subject patient can be predicted before surgery, or a status of the subject patient can be predicted after surgery to generate guide information. At this time, the deep learning method is not limited, and at least one method may be applied according to circumstances (necessity). Here, as an example of an artificial intelligence algorithm, a Recurrent Neural Network (RNN) or a transformer may be applied, but it is not limited thereto, and other artificial intelligence algorithms may be applied.

In the present specification, the description has been limited to the ‘providing apparatus 100’, but this is a medical staff terminal (including a manager terminal, a medical institution terminal, etc.) for generating and providing guide information according to a status of a subject patient predicted based on artificial intelligence, and may include all various devices capable of performing computational processing. That is, the providing apparatus 100 may further include or be in the form of any one of a server, a computer, and a portable terminal, and is not limited thereto.

Here, the computer may include, for example, a laptop, a desktop, a tablet PC, a slate PC, and the like, on which a web browser is mounted.

The server is a server that processes information by performing communication with an external device, and may include an application server, a computing server, a database server, a file server, a game server, a mail server, a proxy server, a web server, and the like.

The portable terminal is, for example, a wireless communication device for which portability and mobility are guaranteed, and may include all types of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminals, and smartphones, and wearable devices such as watches, rings, bracelets, anklets, necklaces, glasses, contact lenses, or head-mounted-devices (HMD).

In the present specification, ‘medical data’ includes personal information and medical records (including medical history, surgical history, prescription history, medical images, etc.) of a subject patient, and may be collected from the medical staff terminal 200 as well as devices (servers, terminals, etc.) connected to various other management agencies, managers, medical staff, medical institutions, and the like. In this case, the medical data includes time-series data for a certain period.

First, the present disclosure is intended to provide prediction information to a medical staff terminal by predicting a probability of occurrence of a postoperative complication for a surgical site of a patient before surgery, so that the medical staff considers the prediction information during surgery. In addition, after surgery, an image captured by the patient himself/herself easily through a user terminal, which is his/her own terminal, is used to analyze a joint range of motion and a degree of muscular strength using an artificial-intelligence-based pre-trained model, and a rehabilitation exercise progress level and a target value for the subject patient are compared, thereby generating and providing customized guide information suitable for the subject patient.

Hereinafter, operation principles and embodiments of the present disclosure will be described with reference to the accompanying drawings.

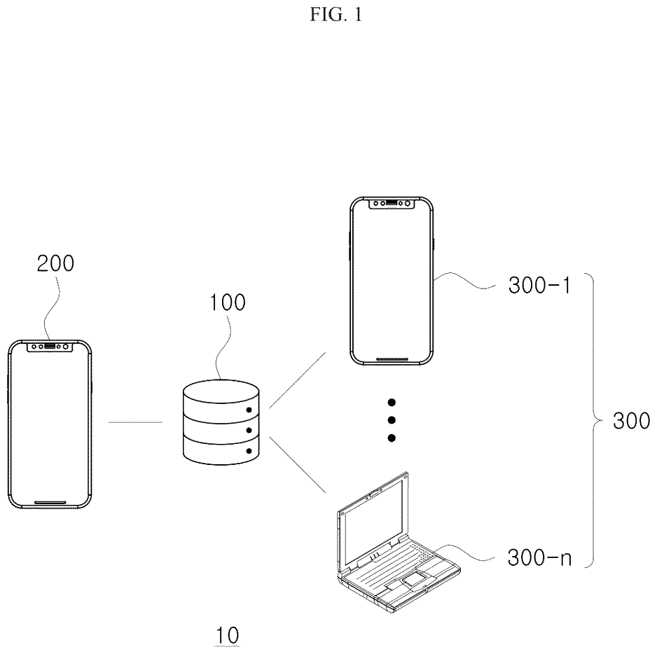

FIG. 1 is a diagram illustrating a system for providing guide information for rehabilitation exercise based on artificial intelligence according to an embodiment of the present disclosure.

Referring to FIG. 1, a system 10 for providing guide information for rehabilitation exercise based on artificial intelligence (hereinafter referred to as a ‘providing system’) according to an embodiment of the present disclosure may be configured to include a providing apparatus 100, a medical staff terminal 200, and a user terminal 300. At this time, although one medical staff terminal 200 and one user terminal 300 are shown in FIG. 1, each may be composed of at least one or more, and the number and type thereof are not limited.

First, the providing apparatus 100 collects medical data and medical images captured before surgery of a subject patient based on the storage module 130 and a separate database, and predicts a probability of occurrence of a postoperative complication by performing preprocessing on the medical images and inputting them to a first artificial-intelligence-based pre-trained model. Accordingly, the providing apparatus 100 may generate prediction information based on the prediction result and provide it to the medical staff terminal 200.

Furthermore, in the case that a rehabilitation exercise video of the subject patient captured after surgery is received from the user terminal 300, the providing apparatus 100 performs preprocessing on the rehabilitation exercise video and inputs it into a second artificial-intelligence-based pre-trained model to check rehabilitation exercise progress information according to a joint range of motion and a degree of muscular strength of the subject patient. Accordingly, the providing apparatus 100 may generate guide information based on the rehabilitation exercise progress information and provide it to the user terminal 300.

Meanwhile, the medical staff terminal 200 is a terminal possessed by a medical staff, a medical institution, a manager, a management agency, or the like, who records and manages medical data (including medical images) for each of at least one patient, and may determine whether to provide the medical data according to a request from the providing apparatus 100, and may provide the medical data to various agencies as well as the providing apparatus 100 as necessary.

In addition, the medical staff terminal 200 may receive prediction information generated by the providing apparatus 100 based on the provided medical data of the subject patient, and may consider the prediction information when performing surgery on the subject patient.

Meanwhile, the user terminal 300 refers to a user who has authority to transmit a rehabilitation exercise video of a patient to the providing apparatus 100 or receive guide information from the providing apparatus 100 as a terminal possessed by the subject patient himself/herself as well as at least one or more guardians preset in response to the patient. That is, the user terminal 300 is a concept including a patient terminal and a guardian terminal, and may be pre-registered by the providing apparatus 100 or the medical staff terminal 200 so that access to the providing apparatus 100 is possible.

Meanwhile, the medical staff terminal 200 and the user terminal 300 may be a computer, UMPC (Ultra Mobile PC), workstation, net-book, PDA (Personal Digital Assistants), portable computer, web tablet, wireless phone, mobile phone, smartphone, pad, smart watch, wearable terminal, e-book, PMP (portable multimedia player), portable game console, navigation device, black box, digital camera, or other mobile communication terminals on which various application programs desired by a medical staff (including a medical institution, manager, management agency) and/or a user (including a patient, guardian) may be installed and executed. That is, the medical staff terminal 200 and the user terminal 300 may be provided in various forms, and this is not limited.

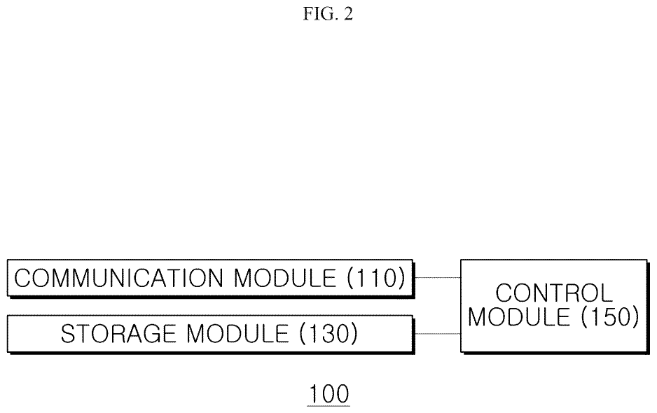

FIG. 2 is a diagram illustrating a configuration of an apparatus for providing guide information for rehabilitation exercise based on artificial intelligence according to an embodiment of the present disclosure.

Referring to FIG. 2, the apparatus 100 for providing guide information for rehabilitation exercise based on artificial intelligence (hereinafter referred to as the ‘providing apparatus’) according to an embodiment of the present disclosure may be configured to include a communication module 110, a storage module 130, and a control module 150.

The communication module 110 transmits and receives at least one piece of information or data to and from at least one device/terminal. Here, the at least one device/terminal may be a device/terminal to be provided with artificial-intelligence-based prediction information or guide information, or a device/terminal that provides various data/information so that it may be generated, and the type and form thereof are not limited.

In addition, this communication module 110 may perform communication with other devices and transmits and receives wireless signals in a communication network according to wireless Internet technologies.

Wireless Internet technologies include, for example, WLAN (Wireless LAN), Wi-Fi (Wireless-Fidelity), Wi-Fi Direct, DLNA (Digital Living Network Alliance), WiBro (Wireless Broadband), WiMAX (World Interoperability for Microwave Access), HSDPA (High Speed Downlink Packet Access), HSUPA (High Speed Uplink Packet Access), LTE (Long Term Evolution), LTE-A (Long Term Evolution-Advanced), etc., and the providing apparatus 100 transmits and receives data according to at least one wireless Internet technology within a range including Internet technologies not listed above.

For short-range communication, short-range communication may be supported using at least one of Bluetooth™, RFID (Radio Frequency Identification), Infrared Data Association (IrDA), UWB (Ultra Wideband), ZigBee, NFC (Near Field Communication), Wi-Fi, Wi-Fi Direct, and Wireless USB (Wireless Universal Serial Bus) technologies. Such Wireless Local Area Networks may support wireless communication between the providing apparatus 100 and the medical staff terminal 200 and/or the user terminal 300. At this time, the Wireless Local Area Network may be a Wireless Personal Area Network.

The storage module 130 may store at least one process (algorithm) for providing artificial-intelligence-based prediction information or guide information or data for a program that reproduces the process. In addition, the storage module 130 may further store processes for performing other operations, and is not limited thereto.

The storage module 130 may store various data supporting various functions of the providing apparatus 100 as well as medical data for at least one patient. The storage module 130 may store a plurality of application programs (applications) driven in the providing apparatus 100, data for operation of the providing apparatus 100, and instructions. At least some of these application programs may be downloaded from an external server through wireless communication. Meanwhile, an application program is stored in at least one memory provided in the storage module 130, installed on the providing apparatus 100, and may be driven to perform operations (or functions) by at least one processor stored in the storage module 130 through the control module 150.

Meanwhile, the at least one memory may include at least one type of storage medium among flash memory type, hard disk type, multimedia card micro type, card type memory (e.g., SD or XD memory, etc.), RAM (Random Access Memory), SRAM (Static Random Access Memory), ROM (Read-Only Memory), EEPROM (Electrically Erasable Programmable Read-Only Memory), PROM (Programmable Read-Only Memory), magnetic memory, magnetic disk, and optical disk. In addition, the memory may store information temporarily, permanently, or semi-permanently, and may be provided as an internal type or a removable type.

The storage module 130 may build a database for providing artificial-intelligence-based prediction information or guide information, or may be linked with a separate external server.

The control module 150, in addition to operations related to application programs, controls all components within the providing apparatus 100 based on at least one processor to process input or output signals, data, information, etc., or executes instructions, algorithms, and application programs stored in at least one memory to perform various processes, and may provide or process appropriate information or functions for providing artificial-intelligence-based prediction information or guide information.

Specifically, the control module 150 collects medical data and medical images captured before surgery of a subject patient based on the storage module 130 or a separate database, and after performing preprocessing on the medical images, inputs the preprocessed medical images into a first artificial-intelligence-based pre-trained model to predict a probability of occurrence of a postoperative complication, and may generate prediction information based on the prediction results and provide it to the medical staff terminal 200. Here, the medical image is an image captured for the subject patient for a certain period (preset period), and may be, for example, an image acquired through X-ray, CT (Computer Tomography), or MRI (Magnetic Resonance Imaging). At this time, the medical images may include images acquired for the subject patient for a certain period in time-series order.

To this end, the control module 150 may build training data for the first pre-trained model and perform pre-training based thereon. At this time, the control module 150 may preprocess at least one medical image for each of other patients collected for a preset period and build the preprocessed at least one medical image as training data. Thereafter, training may be performed by inputting the training data into the first pre-trained model.

At this time, in the case that the control module 150 performs preprocessing on at least one medical image, it identifies at least one region in each of the at least one medical images, and labels, for each identified at least one region, at least one of whether a postoperative complication occurs and a time of occurrence of the postoperative complication based on the medical data of each of the other patients.

Accordingly, the control module 150 may predict a probability of occurrence of a postoperative complication at a surgical site before surgery based on the medical image of the subject patient, and may generate prediction information as a result of the prediction. Here, the surgical site refers to a part corresponding to each joint of the body and may include a shoulder joint, an elbow joint, a hand joint, a hip joint, a knee joint, a foot/ankle joint, and a spinal joint.

Meanwhile, in the case that a rehabilitation exercise video of the subject patient captured after surgery is received from the user terminal 300, the control module 150 analyzes a motion based on the medical data and the rehabilitation exercise video of the subject patient through a second artificial-intelligence-based pre-trained model, and based on the analysis result, may generate guide information including information on at least one rehabilitation exercise corresponding to the relevant time point and provide it to the user terminal 300. Here, the rehabilitation exercise video is a video including the subject patient performing rehabilitation exercise and may include time-series data regarding joint movement.

To this end, the control module 150 may build training data for the second pre-trained model and perform pre-training based thereon. At this time, the control module 150 may preprocess at least one rehabilitation exercise video for each of other patients collected for a preset period and build the preprocessed at least one rehabilitation exercise video as training data. Thereafter, training may be performed by inputting the training data into the second pre-trained model.

At this time, in the case that the control module 150 performs preprocessing on the at least one rehabilitation exercise video, it classifies the at least one rehabilitation exercise video by rehabilitation stage, and labels a joint range of motion and a degree of muscular strength in each of the classified rehabilitation exercise videos based on pre-stored average target information.

Accordingly, the control module 150 may predict a status (rehabilitation exercise progress level) of the subject patient after surgery based on the rehabilitation exercise video of the subject patient and may generate guide information as a result of the prediction. Here, the guide information may include at least one of a type, a method, an intensity, a duration, and a number of repetitions for each of the at least one rehabilitation exercises that the subject patient should perform to reach a target value.

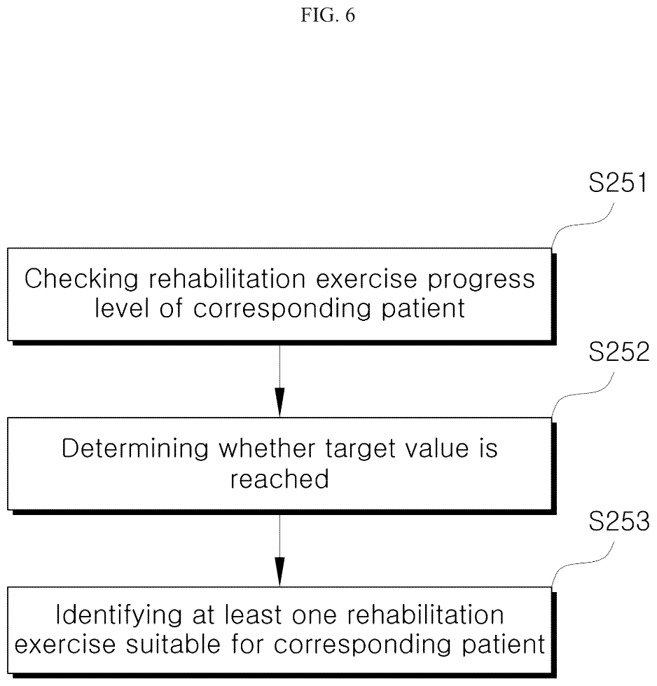

Meanwhile, regarding the operation of the control module 150 for analyzing motion based on a rehabilitation exercise video, it will be described in detail below based on FIG. 6.

In addition, specific operations of the control module 150 will be described below based on each drawing.

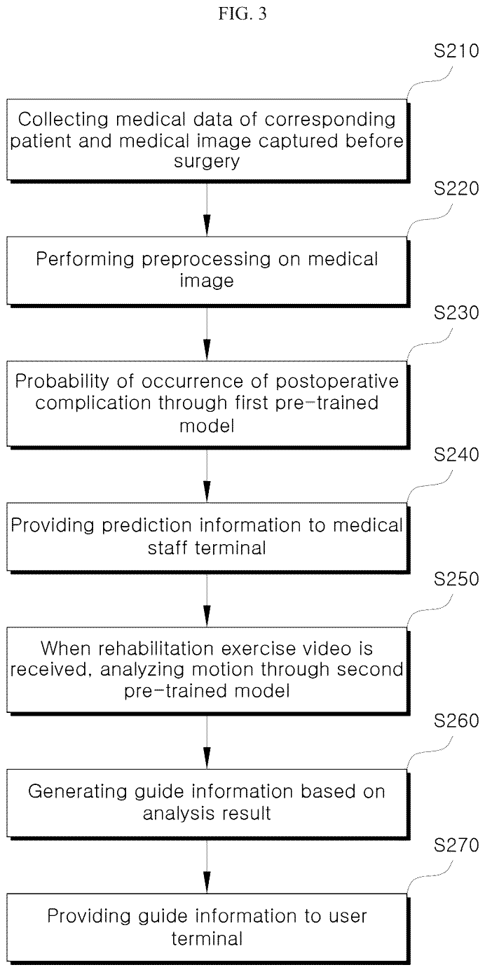

FIG. 3 is a diagram illustrating a method for providing guide information for rehabilitation exercise based on artificial intelligence according to an embodiment of the present disclosure.

Referring to FIG. 3, the providing apparatus 100 collects medical data and medical images captured before surgery of a subject patient based on the storage module 130 or a separate database (S210), and performs preprocessing on the medical images (S220). Here, although medical data and medical images have been described separately, the medical image may be a concept included in medical data, and is not limited thereto.

Next, the providing apparatus 100 inputs the preprocessed medical images into a first artificial-intelligence-based pre-trained model to predict a probability of occurrence of a postoperative complication (S230), and generates prediction information based on the prediction result and provides it to the medical staff terminal 200 (S240).

Next, in the case that a rehabilitation exercise video of the subject patient captured after surgery is received from the user terminal 300, the providing apparatus 100 analyzes a motion based on the medical data and the rehabilitation exercise video of the subject patient through a second artificial-intelligence-based pre-trained model (S250), and generates guide information including information on at least one rehabilitation exercise corresponding to the relevant time point based on the analysis result (S260).

Next, the providing apparatus 100 provides the guide information generated in step S260 to the user terminal 300. At this time, the user terminal 300 may be a terminal possessed by at least one preset patient or guardian, and the number and type thereof are not limited.

Steps S210 to S240 described above relate to pre-surgical operations, and prediction information generated as a result of their execution is provided to the medical staff terminal 200, and steps S250 to S270 relate to post-surgical operations, and guide information generated as a result of their execution is provided to the user terminal 300. Both of these operations do not need to be performed continuously and may be performed separately.

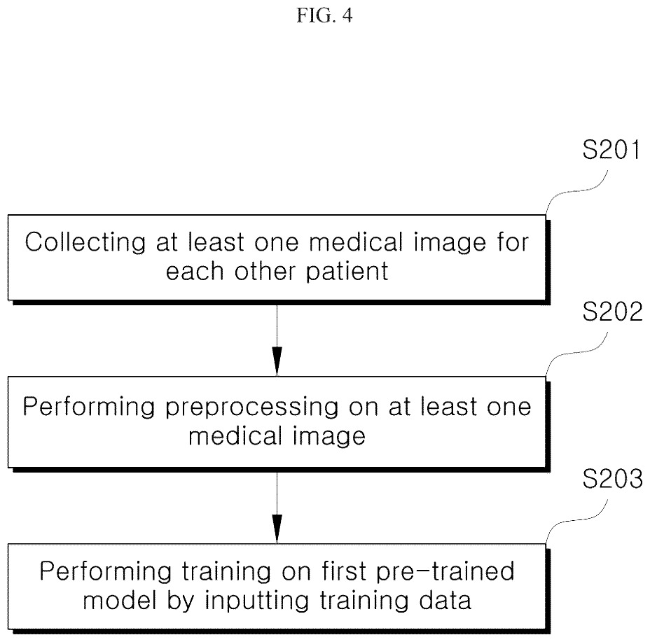

FIG. 4 is a diagram illustrating a method for performing training on a first artificial-intelligence-based pre-trained model according to an embodiment of the present disclosure.

Referring to FIG. 4, the providing apparatus 100 collects at least one medical image for each of other patients for a preset period (S201), and performs preprocessing on the collected at least one medical image (S202). Here, the at least one medical image includes various images captured for each of the other patients for a preset period in time-series order.

Next, the providing apparatus 100 builds the preprocessed at least one medical image as training data and inputs it into the first pre-trained model to perform training (S203).

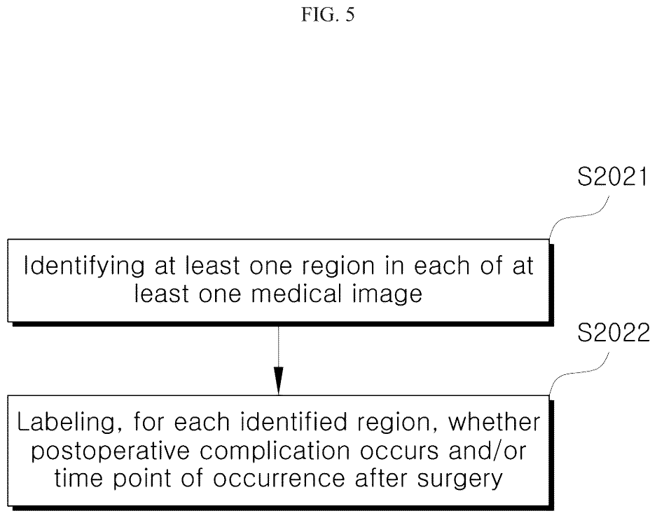

FIG. 5 is a diagram illustrating a series of operations for preprocessing at least one medical image for each of other patients collected according to an embodiment of the present disclosure, which concretely illustrates step S202.

Referring to FIG. 5, the providing apparatus 100 identifies at least one region in each of the at least one medical images (S2021) and labels whether a postoperative complication occurs and/or a time of occurrence of a postoperative complication for each identified at least one region (S2022).

In step S2021 described above, the providing apparatus 100 needs to identify a part to be segmented and identifies it; for example, various regions such as a surface formed by a tendon may be identified.

FIG. 6 is a diagram illustrating a method for analyzing a motion of a subject patient through a second artificial-intelligence-based pre-trained model according to an embodiment of the present disclosure, which more specifically illustrates step S250 of FIG. 3.

Referring to FIG. 6, the providing apparatus 100 measures a joint range of motion and a degree of muscular strength of the subject patient through a rehabilitation exercise video of the subject patient to check a rehabilitation exercise progress level (S251), and determines whether a target value is reached based on pre-stored average target information (S252). At this time, in order to check the rehabilitation exercise progress level in step S251, a gyro sensor provided in the user terminal 200 that captured the rehabilitation exercise video may be further utilized. Meanwhile, when the providing apparatus 100 measures a joint range of motion through a rehabilitation exercise video of the subject patient, it measures the exercise angle in a different manner according to each surgical site (joint part).

Through this, the exercise amount of the subject patient is determined based on the second pre-trained model and compared with pre-stored average target information, thereby determining whether the exercise amount, exercise method, etc., of the subject patient are appropriate, that is, whether they are not insufficient.

At this time, whether the exercise amount, exercise method, etc., of the subject patient are appropriate may be determined differently according to the rehabilitation stage of the subject patient. To this end, the pre-stored average target information may include data regarding joint movement for each part of each rehabilitation stage and information on a target value.

Next, the providing apparatus 100 identifies at least one rehabilitation exercise suitable for the rehabilitation stage or rehabilitation exercise progress level of the subject patient (S253). Here, information for each of the at least one rehabilitation exercises may include at least one of a type, a method, an intensity, a duration, and a number of repetitions for each of the at least one rehabilitation exercises that the subject patient should perform to reach the target value. In other words, at least one rehabilitation exercise mapped to the rehabilitation stage or rehabilitation exercise progress level of the subject patient is identified, and a rehabilitation exercise suitable for the patient to perform is recommended and provided along with the exercise method.

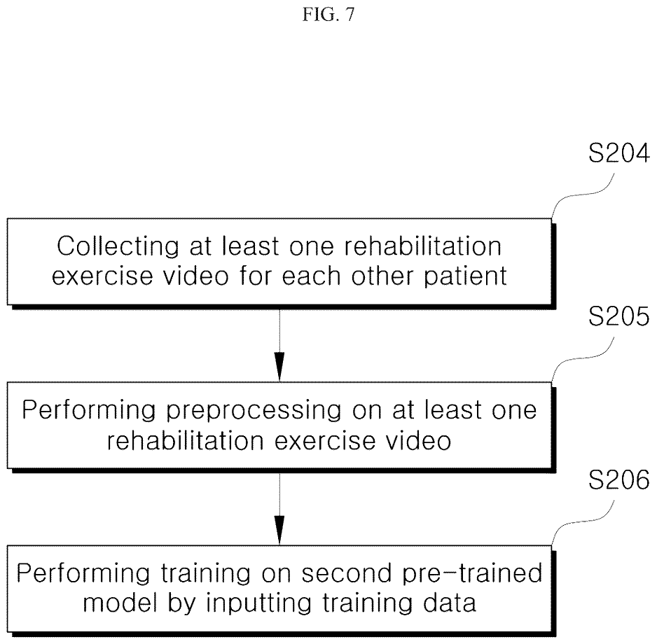

FIG. 7 is a diagram illustrating a method for performing training on a second artificial-intelligence-based pre-trained model according to an embodiment of the present disclosure.

Referring to FIG. 7, the providing apparatus 100 collects at least one rehabilitation exercise video for each of other patients for a preset period (S204), and performs preprocessing on the collected at least one rehabilitation exercise video (S205). Here, the at least one rehabilitation exercise video includes various videos in which other patients perform rehabilitation exercise captured for a preset period in time-series order, and at this time, each rehabilitation exercise video includes joint movement for each patient.

Next, the providing apparatus 100 builds the preprocessed at least one rehabilitation exercise video as training data and inputs it into the second pre-trained model to perform training (S206).

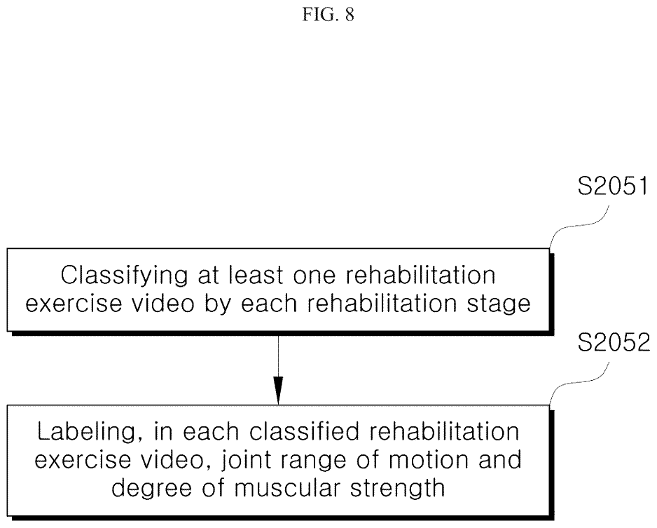

FIG. 8 is a diagram illustrating a series of operations for preprocessing at least one rehabilitation exercise video for each of other patients collected according to an embodiment of the present disclosure, which concretely illustrates step S205.

Referring to FIG. 8, the providing apparatus 100 classifies the at least one rehabilitation exercise video by rehabilitation stage (S2051) and labels a joint range of motion and a degree of muscular strength in each of the classified rehabilitation exercise videos (S2052).

Meanwhile, since the present disclosure performs inference for a predetermined purpose using a model implemented in an artificial neural network method, an artificial neural network will be described below.

A model in the present specification may mean any form of a computer program that operates based on a network function, an artificial neural network, and/or a neural network. Throughout this specification, model, neural network, network function, and neural network may be used interchangeably. In a neural network, one or more nodes are interconnected through one or more links to form an input node and output node relationship within the neural network. Characteristics of a neural network may be determined according to the number of nodes and links within the neural network, the association between nodes and links, and the value of a weight assigned to each of the links. A neural network may consist of a set of one or more nodes. A subset of nodes constituting a neural network may constitute a layer.

A deep neural network (DNN) may mean a neural network including a plurality of hidden layers in addition to an input layer and an output layer, and as a concept thereof is exemplarily shown in FIG. 4, a hidden layer in the middle is composed of one or more, preferably two or more, in a deep neural network.

Such a deep neural network may include a convolutional neural network (CNN), vision transformer, recurrent neural network (RNN), Long Short Term Memory (LSTM) network, GPT (Generative Pre-trained Transformer), auto encoder, GAN (Generative Adversarial Networks), restricted Boltzmann machine (RBM), deep belief network (DBN), Q network, U network, Siamese network, Generative Adversarial Network (GAN), transformer, and the like.

Alternatively, depending on the embodiment, the deep neural network may be a model trained in a transfer learning method. Here, transfer learning refers to a training method in which a pre-trained model (or base part) having a first task is obtained through techniques (MLM and NSP) shown in FIGS. 11 and 12 by pre-training a large amount of unlabeled training data in a semi-supervised or self-learning method, and a target model is implemented by training labeled training data in a supervised learning method in order to fine-tune the pre-trained model to be suitable for a second task. As one of the models trained by such a transfer learning method, there is BERT (Bidirectional Encoder Representations from Transformers), and the like, but it is not limited thereto.

The description of the aforementioned deep neural network is only an example, and the present disclosure is not limited thereto. In the case of the aforementioned convolutional neural network, it consists of a feature learning part that extracts features from an image and a classification part that performs classification using the extracted features. The feature learning part may include a convolutional layer in which features are extracted from an image using a kernel, a ReLU layer which is one of activation functions, and a pooling layer for reducing dimensions of data, but is not limited thereto. In addition, the classification part may include a flatten layer that lines up features extracted from the feature learning part, and a fully connected layer and a softmax function where classification is substantially performed, but is not limited thereto.

A neural network may be trained in at least one method among supervised learning, unsupervised learning, semi-supervised learning, self-supervised learning, or reinforcement learning. Training of a neural network may be a process of applying knowledge for a neural network to perform a specific operation to the neural network.

A neural network may be trained in a direction to minimize errors in output. Training of a neural network is a process of repeatedly inputting training data into a neural network, calculating an error between an output of the neural network for the training data and a target, and backpropagating the error of the neural network from an output layer of the neural network toward an input layer in a direction to reduce the error to update weights of each node of the neural network. In the case of supervised learning, labeled data in which an answer is labeled for each training data is used, and in the case of unsupervised learning, unlabeled data in which an answer is not labeled for each training data may be used. A change amount of a connection weight of each node to be updated may be determined according to a learning rate. Calculation of a neural network for input data and backpropagation of an error may constitute a learning cycle (epoch). The learning rate may be applied differently according to the number of repetitions of the learning cycle of the neural network. In addition, methods such as increase of training data, regularization, dropout for deactivating some nodes, and a batch normalization layer may be applied to prevent overfitting.

Meanwhile, a model disclosed in an embodiment may borrow at least a part of a transformer. A transformer may consist of an encoder that encodes embedded data and a decoder that decodes the encoded data. A transformer may have a structure that receives a series of data and outputs a series of different types of data through encoding and decoding steps. In an embodiment, a series of data may be processed into a form in which a transformer may operate. A process of processing a series of data into a form in which a transformer may operate may include an embedding process. Expressions such as data tokens, embedding vectors, and embedding tokens may refer to data embedded in a form that a transformer may process.

In order for a transformer to encode and decode a series of data, an encoder and decoders within the transformer may process it using an attention algorithm. An attention algorithm may mean an algorithm that, for a given Query, obtains similarity for one or more Keys, reflects the similarity given in this way to a Value corresponding to each Key, and then calculates an attention value by weight-summing the Values to which the similarity is reflected.

Depending on how a query, key, and value are set, various types of attention algorithms may be classified. For example, in the case of obtaining attention by setting a query, key, and value all the same, this may mean a self-attention algorithm. In the case of obtaining attention by reducing dimensions of an embedding vector and obtaining individual attention heads for each divided embedding vector to process a series of input data in parallel, this may mean a multi-head attention algorithm.

In an embodiment, a transformer may consist of modules that perform a plurality of multi-head self-attention algorithms or multi-head encoder-decoder algorithms. In an embodiment, a transformer may also include additional components other than attention algorithms, such as embedding, normalization, and softmax. A method of configuring a transformer using an attention algorithm may include a method disclosed in Vaswani et al., Attention Is All You Need, 2017 NIPS, which is incorporated herein by reference.

A transformer may be applied to various data domains such as embedded natural language, divided image data, and audio waveforms to convert a series of input data into a series of output data. In order to convert data having various data domains into a series of data that may be input to a transformer, a transformer may embed data. A transformer may process additional data representing a relative positional relationship or topological relationship between a series of input data. Alternatively, a series of input data may be embedded by additionally reflecting vectors expressing a relative positional relationship or topological relationship between input data into the series of input data. In an example, a relative positional relationship between a series of input data may include, but is not limited to, word order within a natural language sentence, a relative positional relationship of each divided image, and a time order of divided audio waveforms. A process of adding information representing a relative positional relationship or topological relationship between a series of input data may be referred to as positional encoding.

The aforementioned program may include code coded in a computer language such as C, C++, JAVA, machine language, etc., which may be read by a processor (CPU) of the computer through a device interface of the computer, in order for the computer to read the program and execute the methods implemented as a program. Such code may include functional code related to a function, etc., defining necessary functions for executing the methods, and may include control code related to an execution procedure necessary for the processor of the computer to execute the functions according to a predetermined procedure. In addition, such code may further include memory-reference-related code as to which location (address) in the internal or external memory of the computer additional information or media necessary for the processor of the computer to execute the functions should be referenced. In addition, in the case that the processor of the computer needs to communicate with any other remote computer or server, etc., in order to execute the functions, the code may further include communication-related code as to how to communicate with any other remote computer or server using a communication module of the computer, what information or media should be transmitted and received during communication, etc.

The stored medium refers to a medium that stores data semi-permanently and is readable by a device, rather than a medium that stores data for a short moment, such as a register, cache, and memory. Specifically, examples of the stored medium include ROM, RAM, CD-ROM, magnetic tape, floppy disk, optical data storage device, etc., but are not limited thereto. That is, the program may be stored in various recording media on various servers that the computer may access or in various recording media on the user's computer. In addition, the medium may be distributed in a computer system connected through a network, and computer-readable code may be stored in a distributed manner.

Steps of a method or algorithm described in connection with an embodiment of the present disclosure may be implemented directly in hardware, in a software module executed by hardware, or in a combination of the two. A software module may reside in RAM (Random Access Memory), ROM (Read Only Memory), EPROM (Erasable Programmable ROM), EEPROM (Electrically Erasable Programmable ROM), flash memory, a hard disk, a removable disk, a CD-ROM, or any form of computer-readable recording medium well known in the technical field to which the present disclosure belongs.

The embodiments of the present disclosure have been described with reference to the accompanying drawings, but those skilled in the art to which the present disclosure belongs will understand that the present disclosure can be implemented in other specific forms without changing its technical spirit or essential features. Therefore, it should be understood that the embodiments described above are illustrative in all respects and not restrictive.

According to the present disclosure described above, the probability of occurrence of a complication at a surgical site of a subject patient is predicted based on artificial intelligence so that a medical staff or a medical institution considers the probability of occurrence of the complication when performing surgery or when the patient undergoes postoperative rehabilitation exercise, thereby lowering the probability of occurrence of postoperative complications.

Meanwhile, according to the present disclosure, a patient can self-check a personal status based on artificial intelligence, and customized guide information corresponding to the status is provided, thereby enabling the patient to perform rehabilitation exercise more efficiently and systematically.

The effects of the present disclosure are not limited to the effects mentioned above, and other effects not mentioned will be clearly understood by those skilled in the art from the description.

What is claimed is:

1. An apparatus for providing guide information for rehabilitation exercise based on artificial intelligence, comprising:
a communication module configured to perform communication with an external device;
a storage module configured to store at least one process for providing guide information for rehabilitation exercise based on artificial intelligence; and
a control module configured to perform an operation for providing guide information for rehabilitation exercise based on artificial intelligence based on the at least one process,
wherein the control module is configured to:
collect, based on the storage module and a separate database, medical data of a subject patient and medical images captured before surgery;
perform preprocessing on the medical images, and input the preprocessed medical images to a first artificial-intelligence-based pre-trained model to predict a probability of occurrence of a postoperative complication; and
generate prediction information based on the predicted result and provide the prediction information to a medical staff terminal.

2. The apparatus of claim 1, wherein the first pre-trained model is trained by preprocessing at least one medical image for each of other patients collected for a preset period and inputting the preprocessed at least one medical image as training data to perform training.

3. The apparatus of claim 2, wherein the control module, when performing the preprocessing on the at least one medical image, is configured to:
identify at least one region in each of the at least one medical images; and label, for each of the at least one regions, at least one of whether a postoperative complication occurs and a time of occurrence of the postoperative complication, based on medical data of each of the other patients.

4. The apparatus of claim 1, wherein the control module is configured to:
when a rehabilitation exercise video of the subject patient captured after surgery is received from a user terminal, analyze a motion based on the medical data and the rehabilitation exercise video through a second artificial-intelligence-based pre-trained model, generate the guide information including information on at least one rehabilitation exercise corresponding to a relevant time point based on a result of the analysis, and provide the guide information to the user terminal,
wherein the rehabilitation exercise video includes time-series data regarding joint movement.

5. The apparatus of claim 4, wherein the control module, when analyzing the motion based on the rehabilitation exercise video, is configured to:
measure a joint range of motion and a degree of muscular strength of the subject patient to check a rehabilitation progress level; determine whether a target value is achieved based on pre-stored average target information; and identify the at least one rehabilitation exercise suitable for the subject patient based on a result of the determination.

6. The apparatus of claim 4, wherein the information on each of the at least one rehabilitation exercises includes at least one of a type, a method, an intensity, a duration, and a number of repetitions of each of the at least one rehabilitation exercises that the subject patient is to perform to reach a target value, and
wherein the pre-stored average target information includes data regarding joint movement for each body part in each rehabilitation stage and information on the target value.

7. The apparatus of claim 6, wherein the second pre-trained model is trained by preprocessing at least one rehabilitation exercise video for each of other patients collected for a preset period and inputting the preprocessed at least one rehabilitation exercise video as training data to perform training, and
wherein the control module, when performing preprocessing on the at least one rehabilitation exercise video, is configured to: classify the at least one rehabilitation exercise video by rehabilitation stage; and label, in each of the classified rehabilitation exercise videos, a joint range of motion and a degree of muscular strength based on the pre-stored average target information.

8. A method for providing guide information for rehabilitation exercise based on artificial intelligence, performed by an apparatus, the method comprising:
collecting, based on a storage module and a separate database, medical data of a subject patient and medical images captured before surgery;
performing preprocessing on the medical images;
inputting the preprocessed medical images to a first artificial-intelligence-based pre-trained model to predict a probability of occurrence of a postoperative complication; and
generating prediction information based on the predicted result and providing the prediction information to a medical staff terminal.

9. The method of claim 8, further comprising:
collecting at least one medical image for each of other patients for a preset period;
performing preprocessing on the at least one medical image; and
training the first pre-trained model by inputting the preprocessed at least one medical image as training data.

10. The method of claim 9, wherein performing the preprocessing comprises:
identifying at least one region in each of the at least one medical images; and
labeling, for each of the at least one regions, at least one of whether a postoperative complication occurs and a time of occurrence of the postoperative complication based on medical data of each of the other patients.

11. The method of claim 8, further comprising:
when a rehabilitation exercise video of the subject patient captured after surgery is received from a user terminal, analyzing a motion based on the medical data and the rehabilitation exercise video through a second artificial-intelligence-based pre-trained model; and
generating the guide information including information on at least one rehabilitation exercise corresponding to a relevant time point based on a result of the analysis and providing the guide information to the user terminal,
wherein the rehabilitation exercise video includes time-series data regarding joint movement.

12. The method of claim 11, wherein analyzing the motion based on the rehabilitation exercise video further comprises:
measuring a joint range of motion and a degree of muscular strength of the subject patient to check a rehabilitation progress level;
determining whether a target value is achieved based on pre-stored average target information; and
identifying the at least one rehabilitation exercise suitable for the subject patient based on a result of the determination.

13. The method of claim 11, wherein the information on each of the at least one rehabilitation exercises includes at least one of a type, a method, an intensity, a duration, and a number of repetitions of each of the at least one rehabilitation exercises that the subject patient is to perform to reach a target value, and
wherein the pre-stored average target information includes data regarding joint movement for each body part in each rehabilitation stage and information on the target value.

14. The method of claim 13, further comprising:
collecting at least one rehabilitation exercise video for each of other patients for a preset period;
performing preprocessing on the at least one rehabilitation exercise video; and
training the second pre-trained model by inputting the preprocessed at least one rehabilitation exercise video as training data,
wherein performing the preprocessing comprises:
classifying the at least one rehabilitation exercise video by rehabilitation stage; and
labeling, in each of the classified rehabilitation exercise videos, a joint range of motion and a degree of muscular strength based on the pre-stored average target information.

15. A computer-readable recording medium storing a computer program for executing the method for providing guide information for rehabilitation exercise based on artificial intelligence according to claim 8.
